(19) United States
(12) Patent Application Publication
(10) Pub. No.: US 2003/0069206 A1
(43) Pub. Date: Apr. 10, 2003

(54) METHODS AND KITS FOR ABSORBING FAT

(76) Inventor: Everett J. Nichols, Edmonds, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 09/474,695

(22) Filed: Dec. 29, 1999

Related U.S. Application Data
(63) Continuation-in-part of application No. 09/114,023, filed on Jul. 10, 1998, now Pat. No. 6,130,321.

(51) Int. Cl. A61K 31/722

(52) U.S. Cl. 514/55

(57) ABSTRACT

In one aspect, the present invention provides kits for reducing absorption of lipids by the gastrointestinal tract of a mammalian body. The kits include chitosan, packaging, and instructions indicating that the chitosan may be consumed with food. Chitosan useful for incorporation into kits of the present invention dissolves sufficiently rapidly in gastric juice that it does not have to be consumed in advance of a meal in order to dissolve within the stomach and thereafter effectively entrap lipids in the gastrointestinal tract of the consumer. In a particular embodiment of the kits of the present invention, the chitosan possesses the property of dissolving in a 100-fold (w/w) excess of 0.16N hydrochloric acid, at a temperature in the range of from 16° C. to 25° C., so that the maximum viscosity of the dissolved chitosan solution is reached within 5 minutes after the acid contacts the chitosan. It is desirable to utilize chitosan having a high tap density and/or high bulk density in the kits of the present invention. In another aspect, the present invention provides methods for reducing absorption of lipids by the gastrointestinal tract of a mammalian body. The methods include the step of providing a consumer with a kit including chitosan, packaging, and instructions indicating that the chitosan may be consumed with food. The kits of the present invention are useful in the practice of the methods of the present invention. By utilizing the kits and/or methods for lipid absorption reduction of the present invention a consumer does not have to remember to consume lipid-absorbing chitosan in advance of food consumption.

### RELATED APPLICATIONS

The present application is a continuation-in-part of copending U.S. patent application Ser. No. 09/114,023, filed Jul. 10, 1998 from which benefit of priority is claimed.

### FIELD OF THE INVENTION

This invention relates to chitosan, to kits containing chitosan and to the use of chitosan as a dietary supplement to reduce the absorption of lipids by a mammalian body.

### BACKGROUND OF THE INVENTION

Chitin is a linear polysaccharide composed of β-(1-4)-linked 2-acetamido-2-deoxy-D-glucose units that occurs naturally in the exoskeleton of invertebrates, in particular the carapace of marine crustaceans. Chemical deacetylation of chitin yields chitosan which is a copolymer of 2-amino-2-deoxy-D-glucose and 2-acetamido-2-deoxy-D-glucose units. Chitosan has numerous uses including: an absorbent useful in water purification; a paper wet web strength enhancer; a blood cholesterol lowering compound; a compound useful in adjusting viscosity; a cosmetics additive; a pharmaceutical adjuvant and a dietary supplement.

With respect to the use of chitosan as a dietary supplement, it is known that consumption of chitosan can reduce the blood serum level of lipids, including cholesterol, and promote weight loss by impeding dietary fat absorption in the gastro-intestinal tract. Chitosan is soluble at a pH of less than about pH 6.4, and is therefore soluble in stomach acid. When dissolved chitosan leaves the stomach and enters the duodenum, the stomach acid is neutralized by bicarbonate ions in the duodenum and the chitosan precipitates out of solution. It is believed that during and after precipitation chitosan entraps lipids, thereby preventing their digestion by lipases and absorption through the intestine. Thus, chitosan must dissolve in the stomach acid in order to effectively entrap lipids in the duodenum and subsequent portions of the gastrointestinal tract.

It is generally understood that chitosan preparations should be consumed at least half an hour before a meal in order to provide enough time for the chitosan to fully dissolve in the stomach acid. A person utilizing chitosan as part of a dietary regime to reduce lipid absorption by the gastrointestinal tract must therefore remember to consume the chitosan far enough in advance of each meal to provide time for the chitosan to dissolve.

A high density preparation of chitosan is desirable because, for example, a smaller volume of high density chitosan need be consumed, compared to lower density chitosan, in order to deliver the same dose of chitosan. Thus, for example, a person need consume fewer tablets or capsules of high density chitosan compared to standard preparations of lower density chitosan.

The present inventors have now discovered that the chitosan disclosed in copending U.S. patent application Ser. No. 09/114,023 is readily soluble in acid conditions that mimic gastric juice, as will be further described and characterized herein. Thus, the chitosan disclosed in copending U.S. patent application Ser. No. 09/114,023 does not have to be consumed in advance of consuming food in order to effectively entrap lipids within the gastrointestinal tract of the consumer. Preferably, the chitosan disclosed in copending U.S. patent application Ser. No. 09/114,023 is consumed simultaneously with food, thereby saving the consumer the trouble of remembering to consume chitosan in advance of consuming food.

### SUMMARY OF THE INVENTION

In one aspect, the present invention provides kits for reducing absorption of lipids by a mammalian body, such as a human body. The kits include chitosan, packaging, and instructions indicating that the chitosan may be consumed with food. Chitosan useful for incorporation into kits of the present invention dissolves sufficiently rapidly in gastric juice that it does not have to be consumed in advance of a meal in order to dissolve within the stomach and thereafter effectively entrap lipids in the gastrointestinal tract of the consumer. In a particular embodiment of the kits of the present invention, the chitosan possesses the property of dissolving in a 100-fold (w/w) excess of 0.16N hydrochloric acid (HCL), at a temperature in the range of from 16° C. to 25° C., so that the maximum viscosity of the dissolved chitosan solution is reached within 5 minutes after the acid contacts the chitosan. The viscosity of the chitosan solution can be measured using a Brookfield Viscometer as set forth in Example 8 herein.

It is desirable to utilize chitosan having a high tap density and/or high bulk density in the kits of the present invention. The kits of the present invention preferably include chitosan having a tap density of at least about 0.4 g/ml, more preferably at least about 0.5 g/ml, and having a bulk density of at least about 0.3 g/ml, more preferably at least about 0.4 g/ml, most preferably at least about 0.5 g/ml. Presently preferred chitosans useful for inclusion in the kits of the present invention bind at least 50 g of corn oil per gram of chitosan as determined by the oil-binding assay set forth in Example 9 of the present application wherein the chitosan is contacted with (and at least partially dissolved in) 0.16N hydrochloric acid for 5, 10 or 15 minutes and then precipitated in the presence of corn oil. Examples of chitosans having high tap density and high bulk density (and possessing desirable solubility and fat binding properties) are chitosans treated in accordance with the acid treatment methods described herein, and in copending U.S. patent application Ser. No. 09/114,023 which is incorporated herein by reference.

By way of non-limiting example, the packaging can be a bottle, a plastic bag, or a blister pack. The instructions can be attached to the packaging, for example as a label attached to a bottle. In one representative embodiment of the kits of the present invention, chitosan is packaged within a bottle and the instructions are printed on a label that is affixed to the bottle. In another representative embodiment of the kits of the present invention, chitosan is packaged within a bottle which is packaged within a box, and the instructions are printed on a separate sheet of instructions included within the box.

In another aspect, the present invention provides methods for reducing absorption of lipids by a mammalian body. The methods include the step of providing a consumer with a kit including chitosan, packaging, and instructions indicating that the chitosan may be consumed with food. The kits of the present invention are useful in the practice of the methods of the present invention.

By utilizing the kits and/or methods for lipid absorption reduction of the present invention a consumer does not have to remember to consume lipid-absorbing chitosan in advance of food consumption.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIG. 1 shows the effect of starting chitosan viscosity on the tap density of chitosan treated in accordance with the acid treatment methods disclosed herein.

FIG. 2 shows the effect of the ratio of water to starting chitosan on the tap density of chitosan treated in accordance with the acid treatment methods disclosed herein.

FIG. 3 shows a 50× magnification, scanning electron microscope image of particles of untreated starting chitosan.

FIG. 4 shows a 50× magnification, scanning electron microscope image of particles of chitosan treated in accordance with the acid treatment methods disclosed herein.

### DETAILED DESCRIPTION OF THE PREFERRED EMBODIMENT

As used herein the term “mesh” refers to the size of a particle as determined by its ability to pass through a screen having holes of defined dimensions. The mesh sizes used herein are Tyler equivalents, as set forth in Table 21-12 of the Chemical Engineers' Handbook, Fifth Edition, R. H. Perry and C. H. Chilton, editors. A plus sign before the mesh number indicates that the particles are larger than that mesh size and do not pass through a screen having the indicated mesh size. A minus sign before the mesh number indicates that the particles are smaller than that mesh size and pass through a screen having the indicated, negative mesh size.

The term “chitin” refers to a linear copolymer of N-acetylglucosamine residues and glucosamine residues, wherein greater than 98% of the residues that constitute chitin are N-acetylglucosamine residues.

The term “chitosan” refers to a copolymer of glucosamine residues and N-acetylglucosamine residues having a degree of deacetylation of at least 2%, i.e., at least 2% of the residues that constitute chitosan are glucosamine.

The term “bulk density” refers to the weight of a chitosan sample divided by its non-packed volume. The units of bulk density are grams (g) per cubic centimeter (cc), abbreviated as g/cc, or, alternatively, grams (g) per milliliter (ml), abbreviated as g/ml. In the practice of the present invention, the bulk density of chitosan is typically measured by pouring ground chitosan into a 10 ml graduated cylinder and dividing the weight of the chitosan in the cylinder by its volume.

The term “tap density” refers to the weight of a chitosan sample divided by its packed volume. The tap density is determined by placing a chitosan sample into a 10 ml graduated cylinder and recording the weight of the chitosan. The open end of the graduated cylinder is sealed (for example with a plastic film). The graduated cylinder is then forcefully tapped up and down 300 times on a solid surface until a constant volume is attained, i.e., the volume of the chitosan in the cylinder cannot be reduced further by continued tapping. The weight of the chitosan is divided by its final, constant volume to yield the tap density. The units of tap density are grams (g) per cubic centimeter (cc), abbreviated as g/cc, or, alternatively, grams (g) per milliliter (ml), abbreviated as g/ml.

In one aspect, the present invention provides kits for reducing absorption of lipids by a mammalian body, such as a human body. The kits include chitosan, packaging, and instructions indicating that the chitosan may be consumed with food. The phrase “chitosan may be consumed with food” means that the chitosan may be consumed anytime from within half an hour before consuming food to one hour after consuming food. Preferably, the chitosan is consumed within fifteen minutes of consuming food, more preferably within five minutes of consuming food. Most preferably the chitosan is consumed at the same time as consuming food.

Chitosan useful for incorporation into kits of the present invention dissolves sufficiently rapidly in gastric juice that it does not have to be consumed in advance of a meal in order to dissolve within the stomach and thereafter effectively entrap lipids in the gastrointestinal tract of the consumer. In a particular embodiment of the kits of the present invention, the chitosan possesses the property of dissolving in a 100-fold (w/w) excess of 0.16N hydrochloric acid (HCL), at a temperature in the range of from 16° C. to 25° C., so that the maximum viscosity of the dissolved chitosan solution is reached within 5 minutes after the acid contacts the chitosan. In another embodiment of the kits of the present invention, the chitosan possesses the property of dissolving in a 100-fold (w/w) excess of 0.16N hydrochloric acid (HCL), at a temperature in the range of from 16° C. to 25° C., so that the maximum viscosity of the dissolved chitosan solution is reached within 2 minutes after the acid contacts the chitosan. The viscosity of the chitosan solution can be measured using a Brookfield Viscometer as set forth in Example 8 herein.

It is desirable to utilize chitosan having a high tap density and/or high bulk density in the kits of the present invention. The kits of the present invention preferably include chitosan having a tap density of at least about 0.4 g/ml, more preferably at least about 0.5 g/ml, and having a bulk density of at least about 0.3 g/ml, more preferably at least about 0.4 g/ml and most preferably at least about 0.5 g/ml. Thus, for example, in one embodiment the kits of the present invention include chitosan having a bulk density of at least about 0.3 g/ml and a tap density of at least about 0.4 g/ml. In another representative embodiment, the kits of the present invention include chitosan having a tap density and a bulk density each of at least about 0.4 g/ml. Presently preferred chitosans useful for inclusion in the kits of the present invention bind at least 50 g of corn oil per gram of chitosan after being contacted with (and at least partially dissolved in) 0.16 N hydrochloric acid for 5, 10 or 15 minutes, and then precipitated in the presence of corn oil, in accordance with the oil-binding assay set forth in Example 9 herein. Examples of chitosans having high tap density and high bulk density (and possessing desirable solubility and fat binding properties) are chitosans treated in accordance with the acid treatment methods described herein, and in copending U.S. patent application Ser. No. 09/114,023 which is incorporated herein by reference.

By way of non-limiting example, the kit packaging can be a bottle, a plastic bag, or a blister pack. The chitosan can also be packaged within a plurality of paper bags that are packaged within a box. The instructions can be attached to the packaging, for example as a label attached to a bottle. In one representative embodiment of the kits of the present invention, chitosan is packaged within a bottle and the instructions are printed on a label that is affixed to the bottle. In another representative embodiment of the kits of the present invention, chitosan is packaged within a bottle which is packaged within a box (such as a cardboard box), and the instructions are printed on a separate sheet of instructions included within the box.

Thus, in accordance with the foregoing, and by way of representative example, in one embodiment the present invention provides a kit including chitosan, packaging, and instructions indicating that the chitosan may be consumed with food, wherein the chitosan possesses the property of dissolving in a 100-fold (w/w) excess of 0.16N hydrochloric acid, at a temperature in the range of from 16° C. to 25° C., so that the maximum viscosity of the dissolved chitosan solution is reached within 5 minutes (more preferably within 2 minutes) after the acid contacts the chitosan. In yet another embodiment, and by way of representative example, the present invention provides a kit including chitosan, packaging, and instructions indicating that the chitosan may be consumed with food, wherein the chitosan has a tap density of at least about 0.4 g/ml (more preferably at least about 0.5 g/ml) and binds at least 50 g of corn oil per gram of chitosan as determined by the oil-binding assay set forth in Example 9 wherein the chitosan is contacted with 0.16N hydrochloric acid for 15 minutes (more preferably for 10 minutes, most preferably for 5 minutes) and then precipitated in the presence of corn oil. In a further embodiment, and by way of representative example, the present invention provides a kit including chitosan, packaging, and instructions indicating that the chitosan may be consumed with food, wherein the chitosan (a) possesses the property of dissolving in a 100-fold (w/w) excess of 0.16N hydrochloric acid, at a temperature in the range of from 16° C. to 25° C., so that the maximum viscosity of the dissolved chitosan solution is reached within 5 minutes (more preferably within 2 minutes) after the acid contacts the chitosan, and (b) binds at least 50 g of corn oil per gram of chitosan as determined by the oil-binding assay set forth in Example 9 wherein the chitosan is contacted with 0.16N hydrochloric acid for 15 minutes (more preferably for 10 minutes, most preferably for 5 minutes) and then precipitated in the presence of corn oil.

In another aspect, the present invention provides methods for reducing absorption of lipids by a mammalian body. The methods include the step of providing a consumer with a kit including chitosan, packaging, and instructions indicating that the chitosan may be consumed with food. The kits of the present invention are useful in the practice of the methods of the present invention.

Thus, in accordance with the foregoing, and by way of representative example, one embodiment of the methods of the present invention includes the step of providing a consumer with a kit including chitosan, packaging, and instructions indicating that the chitosan may be consumed with food, wherein the chitosan possesses the property of dissolving in a 100-fold (w/w) excess of 0.16N hydrochloric acid, at a temperature in the range of from 16° C. to 25° C., so that the maximum viscosity of the dissolved chitosan solution is reached within 5 minutes (more preferably within 2 minutes) after the acid contacts the chitosan. Another embodiment of the methods of the present invention includes the step of providing a consumer with a kit including chitosan, packaging, and instructions indicating that the chitosan may be consumed with food, wherein the chitosan has a tap density of at least about 0.4 g/ml (more preferably at least about 0.5 g/ml) and binds at least 50 g of corn oil per gram of chitosan as determined by the oil-binding assay set forth in Example 9 wherein the chitosan is contacted with 0.16N hydrochloric acid for 15 minutes (more preferably for 10 minutes, most preferably for 5 minutes) and then precipitated in the presence of corn oil. Yet another embodiment of the methods of the present invention includes the step of providing a consumer with a kit including chitosan, packaging, and instructions indicating that the chitosan may be consumed with food, wherein the chitosan (a) possesses the property of dissolving in a 100-fold (w/w) excess of 0.16N hydrochloric acid, at a temperature in the range of from 16° C. to 25° C., so that the maximum viscosity of the dissolved chitosan solution is reached within 5 minutes (more preferably within 2 minutes) after the acid contacts the chitosan, and (b) binds at least 50 g of corn oil per gram of chitosan as determined by the oil-binding assay set forth in Example 9 wherein the chitosan is contacted with 0.16N hydrochloric acid for 15 minutes (more preferably for 10 minutes, most preferably for 5 minutes) and then precipitated in the presence of corn oil.

Chitosan that is useful in the kits and methods of the present invention can be prepared by an acid treatment method that includes mixing chitosan with an effective amount of water and an effective amount of acid, and drying the mixture of chitosan, water and acid, as more fully described hereafter. Chitosan useful as a starting material (hereinafter referred to as “starting chitosan”) in the foregoing acid treatment method is readily available and is derived from chitin obtained from the exoskeleton of invertebrates, especially marine crustaceans. For example, starting chitosan can be obtained in the following manner. Chitin is first extracted from crustacean shells by soaking the shells in 2M hydrochloric acid at 20° C. to 30° C. for approximately five hours. The demineralized shells are washed with water and then deproteinized by soaking in 1-3% sodium hydroxide at 40° C. to 50° C. for approximately six hours, followed by washing in water. The resulting chitin is then deacetylated by treating with a 50% sodium hydroxide solution at approximately 40° C. to 50° C. for eighteen hours, then washing with water until the pH is approximately 7.0. The resulting starting chitosan can be ground to a desired mesh size.

The molecular mass of starting chitosan should preferably be in the range of from about 1000 Daltons to about 2,000,000 Daltons, most preferably from about 10,000 Daltons to about 1,000,000 Daltons.

It has been found that the particle size of the starting chitosan affects the tap density of the treated chitosan derived therefrom. In general, the smaller the particle size of the starting chitosan, the higher will be the tap density of the treated chitosan derived therefrom. Preferably, the particle size of the starting chitosan is smaller than about 20 mesh, most preferably smaller than about 40 mesh. Chitosan powder can be ground to the desired particle size by any art-recognized means, such as the use of a high speed, turbine-type Jacobson grinder.

It has also been found that the viscosity of the starting chitosan affects the tap density of the treated chitosan derived therefrom. In general, the lower the viscosity of the starting chitosan, the higher will be the tap density of the treated chitosan derived therefrom. Preferably, the starting chitosan should have a viscosity of at least about 1 cps, where the abbreviation “cps” refers to the centipoise unit of viscosity. Typically, viscosity of the starting chitosan is determined using a 1% (w/w) chitosan solution in the following manner. The chitosan is dissolved in 1% (w/w) glacial acetic acid diluted in distilled water. The chitosan solution is prepared for viscosity determination by adding 1 gram of dry chitosan to 98 grams of distilled water followed by 1 gram of glacial acetic acid and stirred for 2 hours at 25° C. Viscosity is determined at the end of the two hour stirring period at 25° C. using a Brookfield Viscometer.

Additionally, it has been found that the degree of deacetylation of the starting chitosan affects the tap density of the treated chitosan derived therefrom. The starting chitosan will typically have a percent deacetylation of from about 65% to about 95%, preferably a percent deacetylation of from about 75% to about 90%. Chitin can be deacetylated to produce starting chitosan by any art-recognized means, such as by treating chitin with a solution of 50% sodium hydroxide at a temperature of 40° C., or higher. The process of deacetylation occurs more quickly and efficiently at higher temperature.

Starting chitosan is mixed with water containing at least one acid. The amount of water added to the starting chitosan affects the tap density of the treated chitosan derived therefrom. The amount of water added to the starting chitosan should be adjusted to allow for the water already present in the starting chitosan, which should preferably be dried to a moisture content of from 0% water to about 20% water. The weight of water added to the starting chitosan should not exceed about ten times the weight of the starting chitosan. Preferably the weight of water added to the starting chitosan is about five times the weight of the starting chitosan. If starting chitosan is mixed with an amount of acid that weighs less than the starting chitosan, and an amount of water that is equal to 10 times or more of the weight of the starting chitosan, then high tap density will not be obtained. Conversely, if starting chitosan is mixed with an amount of acid that weighs more than the starting chitosan, and an amount of water that is 10 times or more the weight of the starting chitosan, then the chitosan will dissolve and form a viscous solution and not remain particulate.

Similarly, the amount of acid added to the starting chitosan affects the tap density of the treated chitosan derived therefrom. The weight of acid added to the starting chitosan should be at least about 0.1% of the weight of the starting chitosan that has been dried to a moisture content of from 0% to about 20%. The amount of acid added to the starting chitosan can be adjusted in proportion to the amount of water remaining in the starting chitosan. If an amount of acid is used that approaches, equals or exceeds the weight of the starting chitosan, then the amount of water added to the starting chitosan should be reduced to prevent the chitosan from completely dissolving.

Numerous acids are useful to treat starting chitosan, including, but not limited to: succinic, adipic, hydrochloric, glutamic, lactic, aspartic, acetic, pyruvic, nicotinic and malic. The most effective acids are organic acids that contain at least one carboxyl group. The presently preferred acids are succinic acid, pyruvic acid, malic acid, L-glutamic acid and ascorbic acid.

The starting chitosan is mixed with water and at least one acid for a period of at least one minute, or until the chitosan, water and acid form a homogeneous paste. The water can be at ambient temperature, or can be heated. The treated chitosan is then dried to a moisture content of from 0% to about 20%. Drying can be by any art-recognized means, such as air drying, vacuum drying, heat drying or freeze drying, although the highest chitosan densities are achieved by drying with heat.

An additional, optional step is to neutralize the acid in the chitosan/water/acid paste by adding the paste, before it is dried, to a neutralization bath containing water and a base such as, for example, sodium hydroxide, potassium hydroxide or ammonium hydroxide. The amount of base present is preferably in about ten-fold molar excess of the amount of acid present in the paste. The total volume of the neutralization solution should be sufficient to ensure that the chitosan paste remains sufficiently fluid to permit thorough mixing with the base. Preferably the volume of the neutralization solution is about 10 times the volume of the chitosan paste. The mixture of chitosan, water, acid and base is stirred, the duration of the stirring period is principally determined by the temperature of the mixture. In general, the higher the temperature, the shorter the duration of the stirring period. Thus, for example, at an ambient temperature of from about 20° C. to about 30° C., stirring should continue for at least 18 hours.

The neutralized paste is then preferably rinsed extensively with water in order to remove or wash out the salt of the acid (e.g., sodium succinate where the acid utilized is succinic acid, and the neutralizing base is sodium hydroxide). Alternatively, the neutralization step can be performed after the paste is dried. The dried paste is added to a neutralization bath as described above and stirred for at least 18 hours at ambient temperature, or for a shorter time period at higher temperature. If a base neutralization step is included in the process, a higher concentration of acid can be used to treat the chitosan.

The particle size of the treated chitosan may be reduced in size, preferably by grinding, until a desired particle size is achieved. Thus, for example, a particle size of smaller than 20 mesh, preferably smaller than 40 mesh, is presently preferred for chitosan that is to be used as a dietary supplement, such as chitosan included in the kits of the present invention.

The treated, ground chitosan is preferably then sieved by passing it through stacked sieves, with the mesh size decreasing from the top of the stack to the bottom of the stack, i.e., the size of the apertures in the mesh decreases from the top of the stack to the bottom of the stack. The stacked sieves are constantly vibrated so that the chitosan particles pass through the sieves until each particle encounters a sieve which has a mesh size that is too small to permit the particle to pass through. Alternatively, when it is desirable to obtain only chitosan that has a particle size that is smaller than a specified mesh size, then all the chitosan that passes through a single screen having the specified mesh size is collected.

Chitosan utilized in the kits and methods of the present invention can be encapsulated or tabletized either alone or in combination with other ingredients including vitamins C, E, B6, β-carotene, folic acid, nicotinic acid, antioxidants, lecithin, dietary fiber and a variety of binders and/or lubricants. For the benefit of those individuals who have difficulty swallowing tablets or capsules, chitosan can be added to baked goods, such as crackers, cookies and cakes, and to beverages.

By way of representative example, a person may consume from about 1 gram to about 4 grams of chitosan prior to a meal. Chitosan tablets typically weigh about 1.5 grams and contain about 1.0 gram of chitosan, with binders, lubricants and/or fillers making up the balance of the weight.

The following examples merely illustrate the best mode now contemplated for practicing the invention, but should not be construed to limit the invention.

### EXAMPLE 1

### Preparation of High Tap Density Chitosan Useful in the Practice of the Present Invention

Four samples of starting chitosan were treated as set forth in this Example and the resulting tap density of the treated chitosan samples was determined.

The four chitosan samples were treated under identical conditions. The viscosity of each of the four samples of starting chitosan was determined by using 1% starting chitosan (w/w) solutions in 1% (w/w) acetic acid. Each sample was prepared by adding 1.00 gram of starting chitosan to 98.00 grams of distilled water followed by 1.00 gram of glacial acetic acid and stirred with a magnetic stir bar on a stir plate for a minimum of 2 hours. Viscosity was measured using a Brookfield viscometer.

Starting chitosan samples were all −120 mesh material. 10.00 grams of dry −120 mesh starting chitosan was placed in a beaker. 50.00 grams of distilled water (at an ambient temperature of 20° C. to 30° C.), containing 0.57 grams of dissolved succinic acid, was added to the starting chitosan and the mixture was stirred and mixed with a spatula for approximately 1 to 5 minutes. The resulting pasty/spongy material was spread out on a stainless steel tray to dry at 60° C. for 18 hours in a convection oven.

Following drying, the material was ground in a laboratory grinding mill (Thomas-Wiley) and particles were reduced in size by shearing action. The ground material was screened through an 80 mesh sieve using a laboratory Thomas Sieve Shaker. The material passing through the 80 mesh sieve (Tyler equivalent) was collected and the tap density was determined by placing a chitosan sample into a 10 ml graduated cylinder and recording the weight of the chitosan. The graduated cylinder was then forcefully tapped up and down 300 times on a solid surface until a constant volume is attained, i.e., the volume of the chitosan in the cylinder could not be reduced further by continued tapping. The weight of the chitosan was divided by the final, constant volume to yield the tap density. Aliquots of the untreated, starting chitosan samples were ground and sieved through the same 80 mesh sieve and the tap densities were determined as set forth above on −80 mesh material. The data set forth in Table 1 shows the effect on tap density of treating the four, starting chitosan samples in accordance with the foregoing method.

TABLE 1
Increase in Tap Density Following Treatment

| Starting | Viscosity | Percent | Tap Density | Tap | |
|---|---|---|---|---|---|
| Chitosan | of | Deacetyl- | (g/ml) | Density | Percent |
| Lot | Starting | ation | Untreated, | (g/ml) | Increase |
| (Sample) | Chitosan | of Starting | Starting | Treated | in Tap |
| No. | (cps) | Chitosan | Chitosan | Chitosan | Density |
| 014 | 685 | 77% | 0.294 | 0.506 | 72% |
| 504 | 580 | 75% | 0.316 | 0.489 | 55% |
| 012 | 470 | 73% | 0.352 | 0.538 | 53% |
| 917 | 495 | 75% | 0.316 | 0.519 | 64% |

The results set forth in Table 1 demonstrate that the tap density of the four starting chitosan samples was increased by more than fifty percent by treatment of the starting chitosan as set forth in this Example.

### EXAMPLE 2

### Effect of the Viscosity of the Starting Chitosan

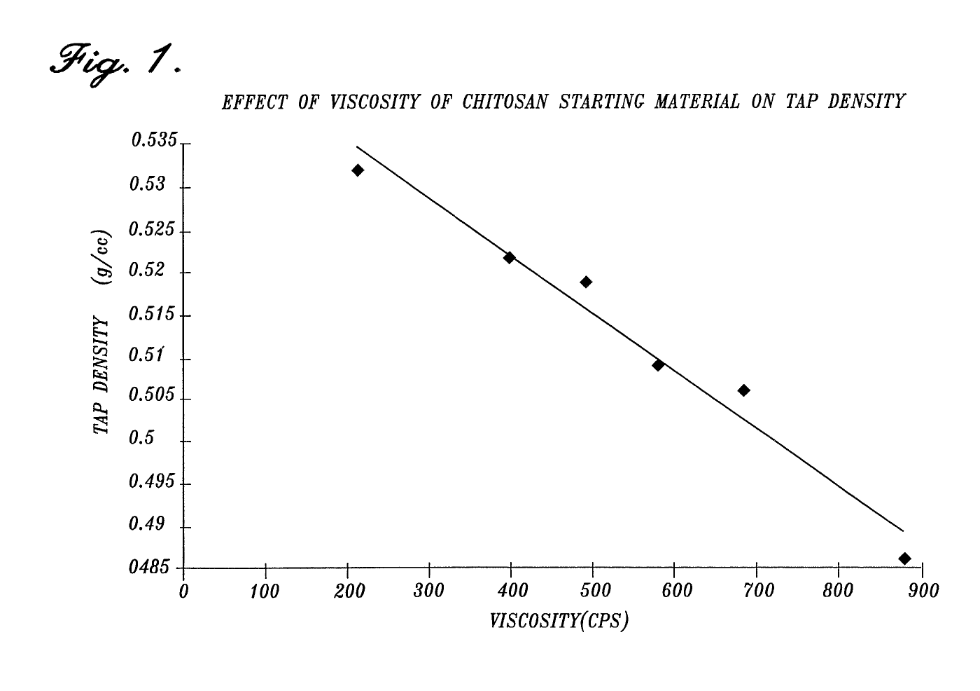

The effect of the viscosity of the starting chitosan on the tap density of the treated chitosan treated in accordance with the acid treatment methods disclosed herein was evaluated in the following manner. Starting chitosan samples were all −120 mesh. The viscosity of the starting chitosan was determined using a Brookfield Viscometer. The treatment conditions were as described in Example 1. In brief, 10 grams of −120 mesh starting chitosan were mixed to a pasty/spongy consistency with 50 grams of water containing 0.57 grams of dissolved succinic acid. The mixture was dried at 60° C., then ground and sieved to yield particles of −80 mesh. Tap densities were determined on −80 mesh material as described in Example 1. The results are shown in FIG. 1.

The results shown in FIG. 1 demonstrate that, in general, the lower the viscosity of the starting chitosan, the higher will be the tap density of the chitosan treated in accordance with the acid treatment methods disclosed herein.

### EXAMPLE 3

### Effect of the Ratio of Water to Starting Chitosan

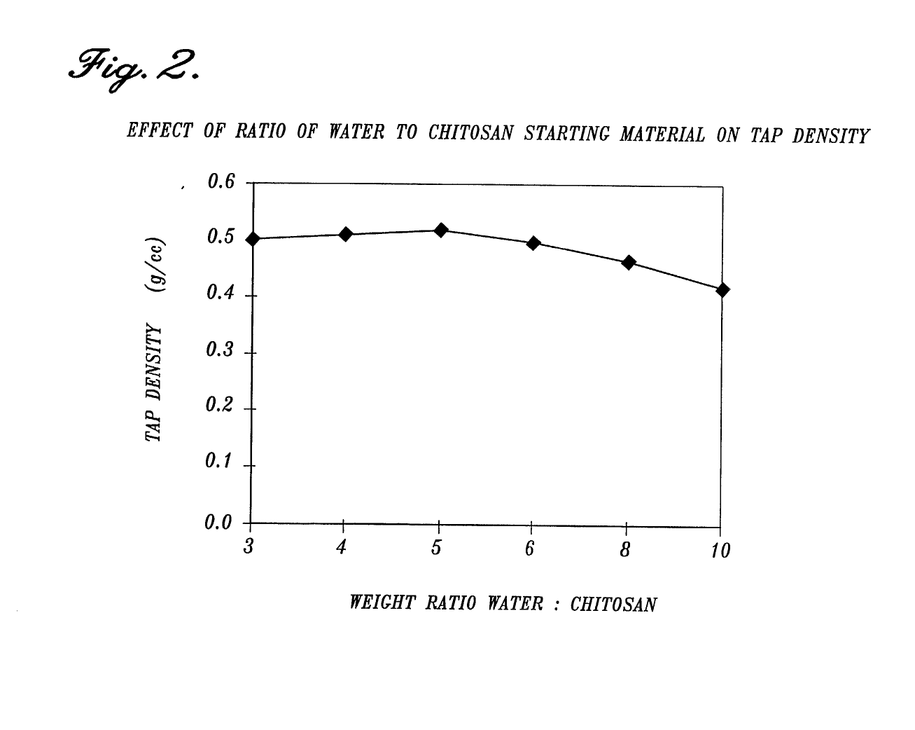

The effect of the ratio of water to starting chitosan on the tap density of chitosan treated in accordance with the acid treatment methods disclosed herein was determined in the following manner. 10 gram aliquots of −120 mesh starting chitosan, all taken from the same lot, were treated with succinic acid in an amount equal to 5.7% of the weight of the starting chitosan. The succinic acid was dissolved in amounts of water equal to 3 times, 4 times, 5 times, 6 times, 8 times or 10 times the weight of the dry, starting chitosan used. For example, 10 grams of starting chitosan were added to 0.57 grams of succinic acid dissolved in 30 grams, or 40 grams, or 50 grams or 60 grams or 80 grams or 100 grams of water. The mixture was mixed to a pasty/spongy consistency, dried at 60° C., ground, sieved through a −80 mesh sieve and tap densities were determined on −80 mesh material. The results of these experiments are set forth in FIG. 2.

### EXAMPLE 4

### The Effect of Various Acids on Chitosan Tap Density

The ability of a variety of acids to increase chitosan tap density when utilized in the acid treatment methods disclosed herein was investigated in the following manner. Since the different acids used in this study differ in molecular weight, the amount of each acid utilized was the molar equivalent of an amount of succinic acid that is 5.7% of the weight of the starting chitosan. If the foregoing molar equivalent amount of acid used was not completely soluble at ambient temperature (20° C. to 30° C.), then the solution was heated to at least 45° C. to dissolve the acid. The heated solution containing the dissolved acid was then added to the starting chitosan and the mixture stirred. The starting chitosan was treated as described in Example 1.

When other acids are used at an equivalent molar concentration to that of succinic acid (used at 5.7% weight of the starting chitosan), these acids are effective at increasing tap density to greater than 0.40 g/cc. The results are shown in Table 2 below.

TABLE 2
Chitosan Tap Density Comparisons Using Various Acids at Molar
Concentrations Equivalent to an Amount of Succinic Acid that
is 5.7% the Weight of the Starting Chitosan

| | | | Treated | |
|---|---|---|---|---|
| Starting | | Molar Equiv. | Chitosan Tap | Treated |
| Chitosan | | to % Succinic | Density | Chitosan |
| Mesh Size | Acid Type | Acid | (grams/cc) | Mesh Size |
| −120 | Control, no | NA | 0.35 | −80 |
| | acid | | | |
| −120 | succinic | 5.7% | 0.58 | −80 |
| −120 | fumaric | 5.7% | 0.52* | −80 |
| −120 | L-glutamic | 5.7% | 0.54* | −80 |
| −120 | hydrochloric | 5.7% | 0.46 | −80 |
| −120 | glucuronic | 5.7% | 0.49 | −80 |
| −120 | citric | 5.7% | 0.42 | −80 |
| −120 | lactic | 5.7% | 0.43 | −80 |
| −120 | malonic | 5.7% | 0.41 | −80 |
| −120 | ascorbic | 5.7% | 0.50 | −80 |
| −120 | aspartic | 5.7% | 0.49* | −80 |
| −120 | acetic | 5.7% | 0.51 | −80 |
| −120 | malic | 5.7% | 0.49 | −80 |
| −120 | pyruvic | 5.7% | 0.52 | −80 |
| −120 | maleic | 5.7% | 0.44 | −80 |

*Solution was heated to dissolve acid.

The data set forth in Table 2 reveal that the acids which are effective to produce chitosan useful in the practice of the present invention include, but are not limited to: succinic, fumaric, hydrochloric, glucuronic, citric, lactic, malonic, ascorbic, aspartic, acetic, malic, pyruvic and maleic.

The acids were also examined at molar equivalent concentrations to an amount of succinic acid that is equal to 28% of the weight of the starting chitosan. If the maximum ambient temperature solubility of the acid was lower than the amount of acid required to provide a molar equivalent to succinic acid (used at 28% of the weight of starting chitosan), then the concentration was defined as a saturated solution of the acid at ambient temperature (20° C. to 30° C.). Heat was not applied to dissolve the acid. The starting chitosan was treated as described in Example 1, except that the mesh size of the treated chitosan was as set forth in Table 3. Results are set forth in Table 3.

TABLE 3
Chitosan Tap Density Comparisons Using Various Acids at Molar
Concentrations Equivalent to an Amount of Succinic Acid
that is 28% the Weight of the Starting Chitosan

| | | Molar Equiv. | Treated | |
|---|---|---|---|---|
| Starting | | to 28% | Chitosan | Final |
| Chitosan | | Succinic | Tap Density | Chitosan |
| Mesh Size | Acid Type | Acid | (grams/cc) | Mesh Size |
| −120 | control, no acid | 0 | not determined | −50 + 80 |
| −120 | control, no acid | 0 | 0.28 | −80 + 120 |
| −120 | control, no acid | 0 | 0.30 | −120 |
| −120 | succinic | 28 | 0.71 | −50 + 80 |
| −120 | succinic | 28 | 0.65 | −80 + 120 |
| −120 | succinic | 28 | 0.59 | −120 |
| −120 | fumaric | saturated sol. | 0.52 | −50 + 80 |
| −120 | fumaric | saturated sol. | 0.42 | −80 + 120 |
| −120 | fumaric | saturated sol. | 0.52 | −120 |
| −120 | glutamic | saturated sol. | 0.29 | −50 + 80 |
| −120 | glutamic | saturated sol. | 0.34 | −80 + 120 |
| −120 | glutamic | saturated sol. | 0.39 | −120 |
| −120 | hydrochloric | 28 | 0.54 | −50 + 80 |
| −120 | hydrochloric | 28 | not determined | −80 + 120 |
| −120 | hydrochloric | 28 | 0.49 | −120 |
| −120 | glucuronic | 28 | 0.67 | −50 + 80 |
| −120 | glucuronic | 28 | 0.65 | −80 + 120 |
| −120 | glucuronic | 28 | 0.68 | −120 |
| −120 | citric | 28 | 0.79 | −50 + 80 |
| −120 | citric | 28 | 0.77 | −80 + 120 |
| −120 | citric | 28 | 0.78 | −120 |
| −120 | lactic (synthetic) | 28 | 0.55 | −50 + 80 |
| −120 | lactic (synthetic) | 28 | not determined | −80 + 120 |
| −120 | lactic (synthetic) | 28 | 0.56 | −120 |
| −120 | lactic (natural) | 28 | 0.58 | −50 + 80 |
| −120 | lactic (natural) | 28 | not determined | −80 + 120 |
| −120 | lactic (natural) | 28 | 0.57 | −120 |
| −120 | malonic | 28 | 0.66 | −50 + 80 |
| −120 | malonic | 28 | 0.41 | −80 + 120 |
| −120 | malonic | 28 | 0.66 | −120 |
| −120 | ascorbic | 28 | 0.71 | −50 + 80 |
| −120 | ascorbic | 28 | 0.68 | −80 + 120 |
| −120 | ascorbic | 28 | 0.68 | −120 |
| −120 | aspartic | saturated sol. | not determined | −50 + 80 |
| −120 | aspartic | saturated sol. | 0.35 | −80 + 120 |
| −120 | aspartic | saturated sol. | 0.39 | −120 |
| −120 | adipic | 28 | 0.55 | −50 + 80 |
| −120 | adipic | 28 | 0.51 | −80 + 120 |
| −120 | adipic | 28 | 0.70 | −120 |
| −120 | acetic | 28 | 0.59 | −50 + 80 |
| −120 | acetic | 28 | not determined | −80 + 120 |
| −120 | acetic | 28 | 0.53 | −120 |
| −120 | malic | 28 | 0.67 | −50 + 80 |
| −120 | malic | 28 | not determined | −80 + 120 |
| −120 | malic | 28 | 0.67 | −120 |
| −120 | pyruvic | 28 | 0.64 | −50 + 80 |
| −120 | pyruvic | 28 | 0.62 | −80 + 120 |
| −120 | pyruvic | 28 | 0.66 | −120 |
| −120 | maleic | 28 | 0.63 | −50 + 80 |
| −120 | maleic | 28 | not determined | −80 + 120 |
| −120 | maleic | 28 | 0.63 | −120 |

When used at saturating concentrations, or the molar equivalent to 28% succinic acid, the majority of the acids tested were effective at increasing the tap density to greater than 0.40 g/ml. The acids not effective were glutamic acid, aspartic and fumaric.

### EXAMPLE 5

### The Effect of Increased Acid and the Addition of a Neutralization Step on Tap Density

When high concentrations of acid are used to treat starting chitosan (such as in the preparation of the high tap density chitosan samples set forth in Table 3), the resulting high tap density chitosan should preferably be treated with a base solution to neutralize the acid. The resulting acid salt can then be rinsed away with water while maintaining the high tap density. The effect on tap density of treating starting chitosan with an increased amount of acid and also incorporating a neutralization step was investigated in the following manner.

Tap densities were determined on dry chitosans ground to smaller than 80 mesh. Neutralization was performed by stirring the dry or wet paste in 1% sodium hydroxide in water for 18 hours at ambient temperature (20° C. to 30° C.) followed by rinsing extensively in water to pH 7. The control treatment consisted of mixing starting chitosan with the same amount of water minus the acid, drying and grinding to smaller than 80 mesh. The amount of water used was 5 times the weight of starting chitosan used. Otherwise, samples were treated as set forth in Example 1. The results are set forth in Table 4.

TABLE 4
Effect of Increased Acid and Neutralization on Tap Density

| Mesh Size of | % Succinic | | Tap Density |
|---|---|---|---|
| Starting Chitosan | Acid | Neutralization | (g/ml) |
| −120 | 28 | none | 0.77 |
| −120 | 28 | before drying | 0.65 |
| −120 | 28 | after drying | 0.76 |
| −120 | 0 (control) | none | 0.35 |
| −50 | 28 | before drying | 0.61 |
| −50 | 28 | after drying | 0.76 |

The data set forth in Table 4 demonstrate that the use of relatively high acid concentrations, and the inclusion of a neutralization step, yields high tap density chitosan.

### EXAMPLE 6

### Bulk Density Values Compared to Tap Density Values for Treated and Untreated Starting Chitosan

Bulk density and tap density were determined using a range of mesh sizes of untreated starting chitosan and chitosan treated in accordance with the acid treatment methods set forth herein. Treated chitosan was treated as set forth in Example 1. The acid used to treat the chitosan samples was succinic acid in an amount equal to 5.7% of the weight of the dry, starting chitosan. Tap density and bulk density were measured in accordance with the methods set forth in the definition of those terms provided herein. The results are set forth in Table 5, below. Chitosan lots K80771012, K50A80210 and K50J80206 are three separate lots of untreated, starting chitosan. Chitosan lot SRW-2398 is succinic acid-treated chitosan.

TABLE 5
Table of Density and Compressibility Index Values

| | | Bulk Density | Tap Density | |
|---|---|---|---|---|
| Mesh Size | Chitosan Lot No. | (gm/cc) | (gm/cc) | C.I. Value |
| +20 | Lot# K80771012 | N/A | N/A | N/A |
| | Lot# K50A80210 | N/A | N/A | N/A |
| | Lot# K50J80206 | N/A | N/A | N/A |
| | Lot# SRW-2398 | 0.47 | 0.51 | 7.8% |
| −20 + 50 | Lot# K80771012 | N/A | N/A | N/A |
| | Lot# K50A80210 | N/A | N/A | N/A |
| | Lot# K50J80206 | N/A | N/A | N/A |
| | Lot# SRW-2398 | 0.46 | 0.52 | 11.5% |
| −50 | Lot# K80771012 | N/A | N/A | N/A |
| | Lot# K50A80210 | 0.20 | 0.29 | 33.3% |
| | Lot# K50J80206 | 0.20 | 0.30 | 33.3% |
| | Lot# SRW-2398 | 0.42 | 0.53 | 21.9% |
| −50 + 80 | Lot# K80771012 | N/A | N/A | N/A |
| | Lot# K50A80210 | 0.21 | 0.25 | 16.0% |
| | Lot# K50J80206 | 0.18 | 0.25 | 28.0% |
| | Lot# SRW-2398 | 0.50 | 0.54 | 7.4% |
| −80 + 120 | Lot# K80771012 | 0.19 | 0.30 | 36.7% |
| | Lot# K50A80210 | 0.18 | 0.26 | 30.8% |
| | Lot# K50J80206 | 0.18 | 0.25 | 28.0% |
| | Lot# SRW-2398 | 0.44 | 0.51 | 13.7% |
| −120 | Lot# K80771012 | 0.19 | 0.32 | 40.6% |
| | Lot# K50A80210 | 0.18 | 0.29 | 37.9% |
| | Lot# K50J80206 | 0.18 | 0.32 | 43.8% |
| | Lot# SRW-2398 | 0.44 | 0.57 | 22.8% |

The C.I. values set forth in Table 5 are Compressibility Index values (also known as Carrs Index values) which are a measure of flowability. The C.I. value is derived by dividing the bulk density by the tap density to yield a fraction which is subtracted from 1, and the resulting value is multiplied by 100. In general, chitosan particles having a Carrs value less than 15 have excellent flow properties; chitosan particles having a Carrs value between 15 and 25 have good flow properties, while chitosan particles having a Carrs value greater than 25 have poor flow properties.

The data set forth in Table 5 demonstrate that the chitosan treated as set forth in this Example has excellent flow properties.

### EXAMPLE 7

### Particle Structure of Chitosan Treated in Accordance with the Method Set Forth in Example 1

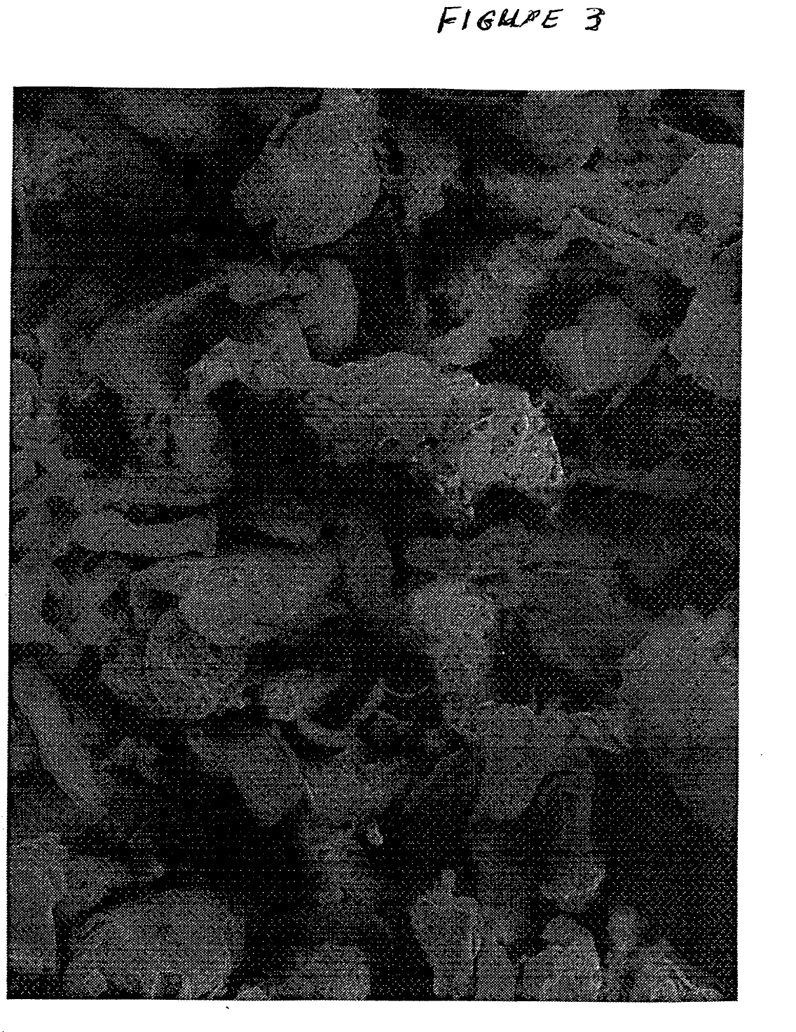

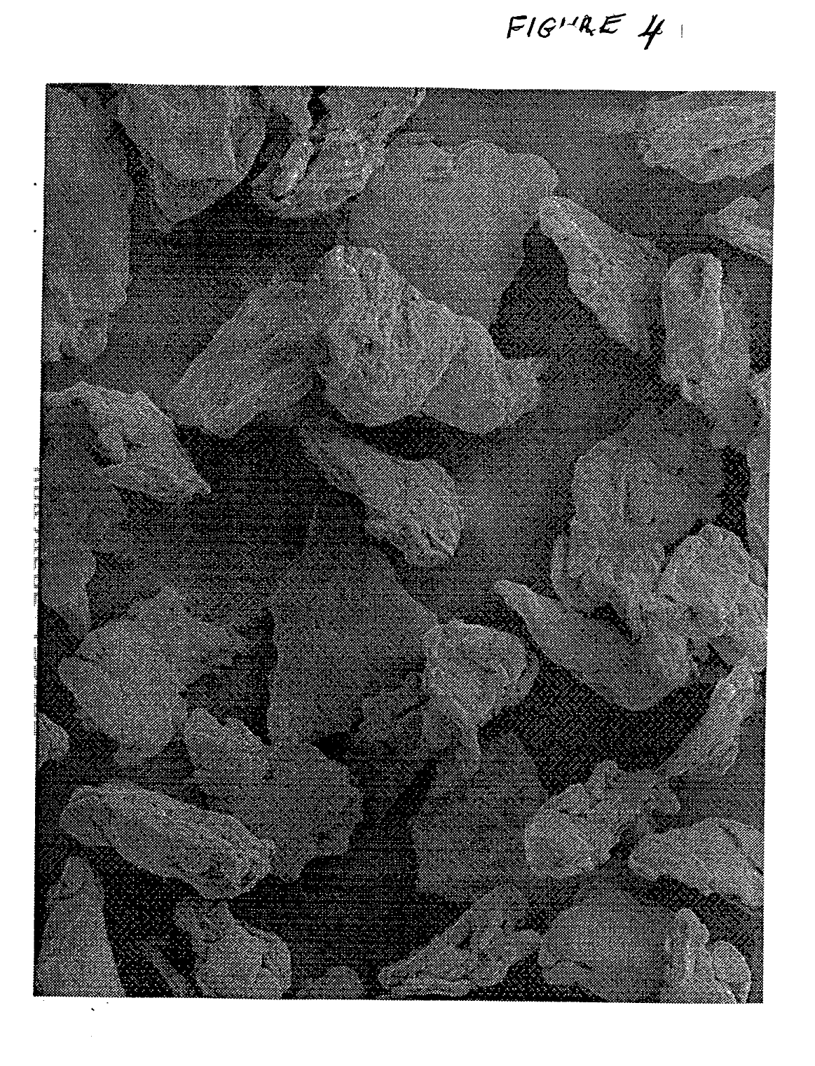

Starting chitosan was treated as set forth in Example 1 and the structure of the treated chitosan particles, and control, untreated, starting chitosan particles, was examined at 50× magnification utilizing a scanning electron microscope. FIG. 3 shows the particle structure of untreated, starting chitosan particles. The particles have an irregular outline and a surface from which fibrous strands project. The untreated chitosan particles also tend to clump together as shown in FIG. 3. In contrast, and as shown in FIG. 4, particles of chitosan treated in accordance with the method set forth in Example 1 herein have a relatively smooth surface and less variation in particle shape compared to untreated, starting chitosan particles. The fibrous projections extending from the untreated, starting chitosan particles are more readily apparent when the particles are viewed under a binocular optical microscope.

### EXAMPLE 8

### Solubility in Hydrochloric Acid of Various Chitosan Samples

The solubility of various chitosan samples, including chitosan treated with succinic acid in accordance with the acid treatment methods set forth in the present application, were determined and compared as follows. All solutions were at ambient temperature (16° C. to 25° C.).

0.16N HCL was prepared in the following way. A clean, dry 500 ml graduated cylinder was three quarters filled with distilled water to which 30.8 ml concentrated HCL (11.7 N) were added. The volume of diluted acid in the graduated cylinder was carefully raised to 500 ml with distilled water and then poured into a clean, dry 2.5 liter (L) container. Three, 500 ml aliquots of distilled water, and one 250 ml aliquot of distilled water, were added to the 500 ml of hydrochloric acid solution in the 2.5 liter container. The final volume of the hydrochloric acid solution was 2.25 L. The final hydrochloric acid concentration was 0.16 N.

200 milliliters (ml) of a 1% (w/w) chitosan solution was prepared in the following manner. The moisture content of the chitosan was first determined by weighing a sample of the chitosan, then drying the sample and weighing the dried sample. The weight of the chitosan sample after drying was divided by the weight of the sample before drying, the resulting fraction was subtracted from 1 and multiplied by one hundred to obtain the percentage of moisture. The percentage of moisture was converted to a decimal (D) by dividing by one hundred. The following equation was used to calculate the weight of chitosan needed to make 200 ml of a 1% (w/w) chitosan solution: Required weight of chitosan=(2.00 g)/(D).

A creased, wax weighing paper was tared on the top loading balance. The required amount of chitosan was weighed to an accuracy of +/−0.01 g and set aside. A 400 ml beaker was tared on the top loading balance. To determine how much HCL solution to pour into the beaker in order to prepare 200 ml of a 1% (w/w) chitosan solution, the following equation was utilized: required amount of 0.16N HCL=[(200.00 g)−(required weight of chitosan)].

The required amount of 0.16N HCL solution (to an accuracy of +/−0.01 g) was added to the tared 400 ml beaker. A 1″×¼″ stir bar was added to the beaker containing the acid which was placed on a stir plate, and stirring was initiated at medium speed to generate a vortex. The rate of rotation of the stir bar was adjusted so that the bottom tip of the vortex lined up with the 125 ml mark on the beaker. The vortex should preferably have a height reaching ˜1″ and the mid-section of the vortex should measure ˜¾″.

Once the vortex had stabilized, the chitosan was gently tapped into the stirring solution. The chitosan was evenly dispersed over the surface of the acid (closer to the wall of the beaker). Each sample of chitosan was consistently added to the acid solution in the same manner. When all of the chitosan was added to the acid solution, the pre-set timer (set for the appropriate mixing time) was started. A hand-held kitchen strainer (approximately 40 mesh), a bowl and cell of a Brookfield Viscometer (with #2 spindle), each clean and dry, were ready to hand. When mixing was completed, the beaker was quickly removed from the stir plate. The contents were filtered through the strainer and the dissolved chitosan solution collected in the bowl. The dissolved chitosan solution was poured from the bowl into the viscometer cell. The viscometer speed was set to 60 rpm, the spindle #2 was positioned in the cell, the viscometer was allowed to run for 4 minutes, and then the resulting values were noted. Viscosity readings were obtained by multiplying the resulting values by a factor of 5. Each sample was run in duplicate and the average values were determined. The average viscosity values are shown in Table 6 wherein HD-EOB is chitosan treated with succinic acid in accordance with the acid treatment methods set forth in the present application and in copending U.S. patent application Ser. No. 09/114,023; HD is a commercially available, high density chitosan having a tap density of about 0.8 g/ml that was not treated with the acid treatment methods described in the present application. Regular chitosan is a commercially available chitosan that has a tap density of about 0.3 g/ml and was not treated with the acid treatment methods described in the present application.

TABLE 6
Time Course Viscosity Comparisons of Various Chitosans

| | Viscosity | | | | | | |
|---|---|---|---|---|---|---|---|
| Chito- | Lot. | | | | | | |
| san | No. | 2 min. | 5 min. | 15 min. | 30 min. | 60 min. | 120 min. |
| HD- | 1011 | 303 cps | 267 cps | 237 cps | 200 cps | 164 cps | 169 cps |
| EOB | | | | | | | |
| HD | 0619 | 297 cps | 292 cps | 264 cps | 288 cps | 258 cps | 214 cps |
| EOB | | | | | | | |
| HD- | 0424 | 229 cps | 229 cps | 220 cps | 204 cps | 177 cps | 159 cps |
| EOB | | | | | | | |
| HD- | 2069 | 82 cps | 88 cps | 80 cps | 73 cps | 73 cps | 75 cps |
| EOB | | | | | | | |
| HD | 0305 | 3 cps | 15 cps | 45 cps | 63 cps | 90 cps | 118 cps |
| Regular | 0311 | 64 cps | 103 cps | 136 cps | 144 cps | 195 cps | 275 cps |
| Regular | 1029 | 22 cps | 54 cps | 129 cps | 154 cps | 146 cps | 140 cps |
| Regular | 0508 | 56 cps | 85 cps | 97 cps | 132 cps | 140 cps | 129 cps |

The maximum viscosity of chitosan HD-EOB is attained within 5 minutes (typically within 2 minutes) of addition to acid, while other chitosans require at least 60 minutes to attain maximum viscosity. It is characteristic of chitosan to decrease in viscosity once full solubilization (maximum viscosity) is reached. The decrease in viscosity is believed to be due to acid hydrolysis of polymer chains.

### EXAMPLE 9

### Fat-Binding Ability of Chitosans Exposed to Acid for Limited Time Periods

The ability of various chitosan samples, including chitosan treated with succinic acid in accordance with the acid treatment methods set forth in the present application, to bind lipids was investigated as follows. All solutions were at ambient temperature (16° C. to 25° C.).

One liter of a solution of 0.16 N HCL was prepared by adding 13.7 ml of concentrated HCL (A.C.S. Reagent grade, 11.7N) to a clean and dry 1 L volumetric flask. Distilled water was slowly added to the HCL until the meniscus just touched the etched line across the neck of the volumetric flask. Filling was stopped periodically to mix the contents by swirling the flask several times.

A 0.2 M carbonate/bicarbonate buffer solution, pH 10.5, was prepared in the following manner. 8.48 g of solid, anhydrous sodium carbonate was dissolved in 400.0 g of distilled water to make Solution A. 6.72 g of solid sodium bicarbonate was dissolved in 400.0 g of distilled water to make Solution B. The 0.2 M carbonate/bicarbonate buffer, pH 10.5, was prepared by mixing Solution A with Solution B in a 9:1 ratio by volume.

Two conical centrifuge tubes were set out for each chitosan sample to be tested. To each of the tubes were added 10 ml of 0.16N HCL and thereafter the tubes were capped. An Orbital Shaker (Lab Line Model 3520) was set up. The shaker had a ¾-inch orbit, generating a horizontal, circular motion, and being capable of a sustained rotation speed of 160 rpm. The sustained rotation speed of 160 rpm was checked before testing each sample by ensuring that the operator could count 80 rotations of the rotor in 30 seconds.

Corn oil (100% corn oil, Mazola brand) and the 0.2M carbonate/bicarbonate buffer were positioned next to the balance ready to be poured immediately. For each sample, the weight of chitosan sample required to provide approximately 0.1 g of chitosan was determined by first calculating the percentage weight loss that occurred upon drying of the chitosan samples (before testing). The percentage weight loss that occurred upon drying was subtracted from one hundred and this new percentage was divided by 100 to convert it to a decimal value (D). To calculate the correct amount of chitosan sample needed, the following equation was used: sample weight needed=(0.1000 g)/D. The required sample weight of chitosan was weighed out to an accuracy of +/−0.0001 g.

One of the centrifuge tubes containing acid was uncapped and placed in a centrifuge tube holder secured to the orbital shaker's platform. The chitosan sample was poured directly onto the surface of the acid solution in the conical centrifuge tube. Care was taken to avoid pouring the chitosan sample down the sides of the tube where it might stick and therefore not come into contact with the acid. Immediately following addition of the chitosan the orbital shaker was run at 160 rpm.

The orbital shaker was run for 2 minutes, 5 minutes or 15 minutes. The centrifuge tubes were removed from the holder as quickly as possible, with a minimum of turbulence, and each tube was tared on a top loading balance. Approximately 10.00 g of corn oil were poured into each tube and brought to exactly 10.00 +/−0.01 g using a transfer pipette (to make pipetting the viscous oil easier with the transfer pipette, the narrow tube tip can be scored with a file and broken off). The weight of each tube containing the chitosan and oil was recorded as Wₛ

Using a 10 ml pipette, 8.5 ml of 0.2M carbonate/bicarbonate buffer, pH 10.5, were immediately added to each tube, the tubes were quickly recapped and vigorously shaken, upside-down, for 30 seconds to neutralize the acid and precipitate the chitosan. Each tube was then placed upright in a conical test tube rack. Each sample was treated in the same way and allowed to sit undisturbed for 30 minutes before being centrifuged at 1,200-1,750×g for exactly two minutes. Spun tubes were removed from the centrifuge and the surface layer of free oil at the top of each tube was pipetted off and weighed to the nearest +/−0.01 g. The weight of free oil was recorded as W_{f}.

The amount of oil bound, or entrapped, per gram of chitosan HD-EOB was calculated according to the following equation wherein the 5% is the correction for 5% (w/w) organic acid content in HD-EOB. Number of grams of entrapped oil per gram of chitosan=Wₛ−W_{f}/(0.1)[1−(% Ash+5%)/100]. The percentage of ash in the chitosan was determined by taking a moisture-corrected sample of chitosan, that includes 2.0 g of chitosan plus the moisture that is present in the sample, and dry ashing the sample in a tared crucible in a muffle furnace at a temperature of approximately 700° C. for 3 hours. The ash remaining in the crucible is weighed and divided by the weight of the chitosan (excluding water in the sample) before ashing (2.0 g) and multiplied by 100 to give the percentage of ash.

The amount of oil bound, or entrapped, per gram of regular or High Density (HD) chitosan is calculated according to the following equation: Number of grams of entrapped oil per gram of chitosan=Wₛ−W_{f}/(0.1)[1−(% Ash)/100]. Note that the 5% correction factor is not used here. However, corrections should be made here for any other additives present in the chitosan. The percentage of other additives, if present, would be added to the % ash.

Table 7 shows a comparison of the fat-binding capacity of various chitosan samples assayed as described in this Example. HD-EOB is chitosan treated with succinic acid as described in accordance with the acid treatment methods set forth in the present application and in copending U.S. patent application Ser. No. 09/114,023; HD is a commercially available, high density chitosan having a tap density of about 0.8 g/ml that was not treated in accordance with the acid treatment methods set forth in the present application. Regular chitosan is a commercially available chitosan that has a tap density of about 0.3 g/ml and was not treated with acid as described in the present application.

TABLE 7
Fat Bindinig Capacity of Various Chitosans

| | | Tap | Time | Oil Binding |
|---|---|---|---|---|
| | | Density | Exposed | grams oil/g |
| Chitosan | Lot No. | g/cc | (min.) | chitosan |
| HD-EOB | 1011 | 0.56 | 2 | 103.8 |
| | | | 5 | 102.8 |
| | | | 15 | 105.9 |
| HD-EOB | 0619 | 0.48 | 2 | 99.8 |
| | | | 5 | 103.8 |
| | | | 15 | 102.2 |
| HD-EOB | 0424 | 0.58 | 2 | 46.2 |
| | | | 5 | 93.1 |
| | | | 15 | 93.3 |
| HD-EOB | 2069 | 0.57 | 2 | 54.9 |
| | | | 5 | 104.2 |
| | | | 15 | 105.5 |
| HD | 0305 | 0.88 | 2 | 6.9 |
| | | | 5 | 2.9 |
| | | | 15 | 24.0 |
| Regular | 0311 | 0.30 | 2 | 7.0 |
| | | | 5 | 8.5 |
| | | | 15 | 13.0 |
| Regular | 1029 | 0.33 | 2 | 7.1 |
| | | | 5 | 8.5 |
| | | | 15 | 11.1 |
| Regular | 0508 | 0.33 | 2 | 8.3 |
| | | | 5 | 8.8 |
| | | | 15 | 45.6 |

The average weight of oil bound by each chitosan sample was calculated by adding the weight of oil bound at 2, 5 and 15 minutes and dividing the sum by three. The average values are shown in Table 8.

TABLE 8
Comparative Oil Binding Over Three Time Points

| Chitosan | Lot No. | Tap Density | Mean Weight of Oil Entrapped. |
|---|---|---|---|
| HD-EOB | 1011 | 0.56 | 104.2 g oil/g chitosan |
| HD-EOB | 0619 | 0.48 | 101.9 g oil/g chitosan |
| HD-EOB | 0424 | 0.58 | 77.5 g oil/g chitosan |
| HD | 0305 | 0.88 | 11.3 g oil/g chitosan |
| Regular | 0508 | 0.33 | 20.9 g oil/g chitosan |
| Regular | 0311 | 0.30 | 9.4 g oil/g chitosan |
| Regular | 1029 | 0.33 | 8.9 g oil/g chitosan |

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A kit for reducing absorption of lipids by the digestive system of a mammalian body, said kit comprising:
(a) chitosan;
(b) packaging; and
(c) instructions indicating that the chitosan may be consumed with food.

2. The kit of claim 1 wherein the chitosan possesses the property of dissolving in a 100-fold (w/w) excess of 0.16N hydrochloric acid, at a temperature in the range of from 16° C. to 25° C., so that the maximum viscosity of the dissolved chitosan solution is reached within 5 minutes after the acid contacts the chitosan.

3. The kit of claim 1 wherein the chitosan possesses the property of dissolving in a 100-fold (w/w) excess of 0.16N hydrochloric acid, at a temperature in the range of from 16° C. to 25° C., so that the maximum viscosity of the dissolved chitosan solution is reached within 2 minutes after the acid contacts the chitosan.

4. The kit of claim 1 wherein the chitosan has a tap density of at least about 0.4 g/ml.

5. The kit of claim 1 wherein the chitosan has a tap density of at least about 0.5 g/ml.

6. The kit of claim 1 wherein the chitosan has a bulk density of at least about 0.4 g/ml.

7. The kit of claim 1 wherein the chitosan has a bulk density of at least about 0.5 g/ml.

8. The kit of claim 1 wherein the chitosan has a bulk density of at least about 0.3 g/ml and a tap density of at least about 0.4 g/ml.

9. The kit of claim 1 wherein the chitosan has a tap density and a bulk density each of at least about 0.4 g/ml.

10. The kit of claim 2 wherein the chitosan has a tap density of at least about 0.4 g/ml.

11. The kit of claim 2 wherein the chitosan has a tap density of at least about 0.5 g/ml.

12. The kit of claim 3 wherein the chitosan has a tap density of at least about 0.4 g/ml.

13. The kit of claim 3 wherein the chitosan has a tap density of at least about 0.5 g/ml.

14. The kit of claim 1 wherein the chitosan has a tap density of at least about 0.4 g/ml and binds at least 50 g of corn oil per gram of chitosan as determined by the oil-binding assay set forth in Example 9 wherein the chitosan is contacted with 0.16N hydrochloric acid for 15 minutes.

15. The kit of claim 1 wherein the chitosan has a tap density of at least about 0.4 g/ml and binds at least 50 g of corn oil per gram of chitosan as determined by the oil-binding assay set forth in Example 9 wherein the chitosan is contacted with 0.16N hydrochloric acid for 10 minutes.

16. The kit of claim 1 wherein the chitosan has a tap density of at least about 0.4 g/ml and binds at least 50 g of corn oil per gram of chitosan as determined by the oil-binding assay set forth in Example 9 wherein the chitosan is contacted with 0.16N hydrochloric acid for 5 minutes.

17. The kit of claim 2 wherein the chitosan has a tap density of at least about 0.4 g/ml and binds at least 50 g of corn oil per gram of chitosan as determined by the oil-binding assay set forth in Example 9 wherein the chitosan is contacted with 0.16N hydrochloric acid for 15 minutes.

18. The kit of claim 3 wherein the chitosan has a tap density of at least about 0.4 g/ml and binds at least 50 g of corn oil per gram of chitosan as determined by the oil-binding assay set forth in Example 9 wherein the chitosan is contacted with 0.16N hydrochloric acid for 15 minutes.

19. The kit of claim 1 wherein the packaging comprises a bottle.

20. The kit of claim 1 wherein the packaging consists of a bottle.

21. The kit of claim 1 wherein the instructions are affixed to the packaging.

22. The kit of claim 19 wherein the instructions are affixed to the bottle.

23. The kit of claim 1 wherein the instructions indicate that the chitosan may be consumed at any time within half an hour before consuming food.

24. The kit of claim 1 wherein the instructions indicate that the chitosan may be consumed at any time within fifteen minutes before consuming food.

25. The kit of claim 1 wherein the instructions indicate that the chitosan may be consumed at any time within five minutes before consuming food.

26. The kit of claim 1 wherein the instructions indicate that the chitosan may be consumed at the same time as consuming food.

27. The kit of claim 1 wherein the instructions indicate that the chitosan may be consumed within an hour after consuming food.

28. A method for reducing absorption of lipids by a mammalian body, the method comprising the step of providing a consumer with a kit, said kit comprising:
(a) chitosan;
(b) packaging; and
(c) instructions indicating that the chitosan may be consumed with food.

29. The method of claim 28 wherein the chitosan possesses the property of dissolving in a 100-fold (w/w) excess of 0.16N hydrochloric acid, at a temperature in the range of from 16° C. to 25° C., so that the maximum viscosity of the dissolved chitosan solution is reached within 5 minutes after the acid contacts the chitosan.

30. The method of claim 28 wherein the chitosan possesses the property of dissolving in a 100-fold (w/w) excess of 0.16N hydrochloric acid, at a temperature in the range of from 16° C. to 25° C., so that the maximum viscosity of the dissolved chitosan solution is reached within 2 minutes after the acid contacts the chitosan.

31. The method of claim 28 wherein the chitosan has a tap density of at least about 0.4 g/ml.

32. The method of claim 28 wherein the chitosan has a tap density of at least about 0.5 g/ml.

33. The method of claim 28 wherein the chitosan has a bulk density of at least about 0.4 g/ml.

34. The method of claim 28 wherein the chitosan has a bulk density of at least about 0.5 g/ml.

35. The method of claim 28 wherein the chitosan has a bulk density of at least about 0.3 g/ml and a tap density of at least about 0.4 g/ml.

36. The method of claim 28 wherein the chitosan has a tap density and a bulk density each of at least about 0.4 g/ml.

37. The method of claim 29 wherein the chitosan has a tap density of at least about 0.4 g/ml.

38. The method of claim 29 wherein the chitosan has a tap density of at least about 0.5 g/ml.

39. The method of claim 30 wherein the chitosan has a tap density of at least about 0.4 g/ml.

40. The method of claim 30 wherein the chitosan has a tap density of at least about 0.5 g/ml.

41. The method of claim 28 wherein the chitosan has a tap density of at least about 0.4 g/ml and binds at least 50 g of corn oil per gram of chitosan as determined by the oil-binding assay set forth in Example 9 wherein the chitosan is contacted with 0.16N hydrochloric acid for 15 minutes.

42. The method of claim 28 wherein the chitosan has a tap density of at least about 0.4 g/ml and binds at least 50 g of corn oil per gram of chitosan as determined by the oil-binding assay set forth in Example 9 wherein the chitosan is contacted with 0.16N hydrochloric acid for 10 minutes.

43. The method of claim 28 wherein the chitosan has a tap density of at least about 0.4 g/ml and binds at least 50 g of corn oil per gram of chitosan as determined by the oil-binding assay set forth in Example 9 wherein the chitosan is contacted with 0.16N hydrochloric acid for 5 minutes.

44. The method of claim 29 wherein the chitosan has a tap density of at least about 0.4 g/ml and binds at least 50 g of corn oil per gram of chitosan as determined by the oil-binding assay set forth in Example 9 wherein the chitosan is contacted with 0.16N hydrochloric acid for 15 minutes.

45. The method of claim 30 wherein the chitosan has a tap density of at least about 0.4 g/ml and binds at least 50 g of corn oil per gram of chitosan as determined by the oil-binding assay set forth in Example 9 wherein the chitosan is contacted with 0.16N hydrochloric acid for 15 minutes.

46. The method of claim 28 wherein the packaging comprises a bottle.

47. The method of claim 28 wherein the packaging consists a bottle.

48. The method of claim 28 wherein the instructions are affixed to the packaging.

49. The method of claim 46 wherein the instructions are affixed to the bottle.

50. A kit for reducing absorption of lipids by the digestive system of a mammalian body, said kit comprising:
(a) chitosan, said chitosan
(1) having a tap density of at least about 0.4 g/ml;
(2) possessing the property of dissolving in a 100-fold (w/w) excess of 0.16N hydrochloric acid, at a temperature in the range of from 16° C. to 25° C., so that the maximum viscosity of the dissolved chitosan solution is reached within 5 minutes after the acid contacts the chitosan; and
(3) binding at least 50 g of corn oil per gram of chitosan as determined by the oil-binding assay set forth in Example 9 wherein the chitosan is contacted with 0.16N hydrochloric acid for 15 minutes;
(b) packaging; and
(c) instructions indicating that the chitosan may be consumed at the same time as food.
